(19) United States
(12) Patent Application Publication
(10) Pub. No.: US 2024/0218456 A1
(43) Pub. Date: Jul. 4, 2024

(54) METHOD FOR RAPID IDENTIFICATION OF HUSO DAURICUS AND ITS HYBRIDS

(71) Applicant: Beijing Academy of Agricultural and Forestry Sciences, Beijing (CN)

(72) Inventors: Hongxia HU, Beijing (CN); Xiaoyu Yan, Beijing (CN); Ying Dong, Beijing (CN); Hua Zhu, Beijing (CN); Tian Dong, Beijing (CN); Wei Wang, Beijing (CN)

(21) Appl. No.: 18/399,643

(22) Filed: Dec. 28, 2023

(30) Foreign Application Priority Data
Dec. 30, 2022 (CN) 202211719841.9

(51) Int. Cl. C12Q 1/6888; C12Q 1/686

(52) U.S. Cl. CPC C12Q 1/6888; C12Q 1/686

(57) ABSTRACT

A method for rapid identification of Huso dauricus and its hybrids provides a DNA molecule of SEQ ID NO: 1 and application of the DNA molecule or more than 10 consecutive nucleotides therein or more than 3 consecutive nucleotides at a terminal thereof as a sturgeon germplasm molecular marker for identifying a sturgeon germplasm of interest, which is Huso dauricus or hybrids of Huso dauricus and other sturgeons. 50 bp InDel mutation region unique to Huso dauricus is screened out. Based on the specific insert fragment, a dominant/co-dominant germplasm-specific molecular marker is developed for identifying Huso dauricus and its hybrids. The dominant molecular marker Hdau216p is used to identify Huso dauricus and hybrids of Huso dauricus blood. According to length specificity of PCR amplification products of Huso dauricus and other sturgeons, co-dominant molecular marker Hdau195l is used to quickly identify purebreds of Huso dauricus and corresponding hybrids by PCR.

### CROSS-REFERENCE TO RELATED APPLICATIONS

The subject application claims priority on Chinese patent application no. 202211719841.9 filed on Dec. 30, 2022 in China. The contents and subject matters of the Chinese priority application is incorporated herein by reference.

### REFERENCE TO AN ELECTRONIC SEQUENCE LISTING

The contents of the electronic sequence listing (Name of the File: ReplacementSequenceListing8026wh.xml; Size: 7,739 bytes; and Date of Creation: Mar. 17, 2024) is herein incorporated by reference in its entirety.

### BACKGROUND OF THE INVENTION

### Technical Field

The invention belongs to the field of biotechnology and relates to a method for rapid identification of Huso dauricus and its hybrids.

### Description of Related Art

Huso dauricus is a cartilaginous fish of the genus Husodauriformes, the family Acipenidae, the order Acipenseriformes. The Huso dauricus has a triangular and relatively pointed snout. Its mouth is located on the ventral surface of the head, with a large cleft and a half-moon shape, and the opercular membrane is not connected to the cheek. The tail is crooked, and there are no scales on the body. There are 5 rows of rhombus-shaped bone plates (hard scales) on the back and both sides. The Huso dauricus can reach 5.6 meters in length and weigh up to 1,000 kilograms. It is distributed in Heilongjiang, the border river between China and Russia.

There are 26 species and subspecies of Acipenseriformes currently in the world, and the main cultured species in my country include more than 5 species including the Huso dauricus. Due to certain hybrid advantages, the hybrid of Huso dauricus (♀) and Acipenser schrenckii (♂) takes an important place in sturgeon breeding in China. There are also hybrids of Huso dauricus and other sturgeons such as Acipenser baeri. According to the restrictions on sturgeon trade in Appendix II of CITES (Convention on International Trade in Endangered Species of Wild Fauna and Flora), the provision of germplasm for caviar and other sturgeon products is a necessary prerequisite for sturgeon trade. The phenomenon of chaotic hybridization in the sturgeon breeding industry has caused great obstacles to the development of China's sturgeon industry. Therefore, accurate sturgeon germplasm identification technology is an important guarantee to support the healthy development of my country's sturgeon industry and the protection of sturgeon germplasm resources.

The existing sturgeon germplasm identification technology is based on morphology and genetics, and also includes molecular marker-assisted identification methods based on mitochondrial DNA and nuclear genes. However, the identification method based on mitochondrial DNA can only identify the female parent in the identification of hybrid sturgeon. The identification of Huso dauricus based on nuclear genome microsatellites, SNPs and other markers has shortcomings such as being unable to completely distinguish Huso dauricus from other sturgeon species, being cumbersome to operate, and being time-extensive for identification. Therefore, the invention screened out rapid identification molecular markers that can accurately distinguish Huso dauricus and its hybrids on the basis of multiple sturgeon genome structure comparison methods.

### SUMMARY OF THE INVENTION

An objective of the invention is to provide a method for rapid identification of Huso dauricus and its hybrids.

The invention provides a DNA molecule, as shown as having a sequence of SEQ ID NO: 1.

The invention further provides an application of the DNA molecule or more than 10 consecutive nucleotides therein or more than 3 consecutive nucleotides at a terminal thereof as a sturgeon germplasm molecular marker; the sturgeon germplasm molecular marker is used to identify a sturgeon germplasm of interest; the sturgeon germplasm of interest is Huso dauricus or a hybrid of Huso dauricus and other sturgeons.

The invention further provides a test primer pair developed on the basis of the DNA molecule or more than 10 consecutive nucleotides therein or more than 3 consecutive nucleotides at a terminal thereof.

The test primer pair may be a dominant primer pair. One primer in the dominant primer pair corresponds to more than 10 consecutive nucleotides in the DNA molecule. Therefore, only the DNA derived from the sturgeon germplasm of interest can be specifically amplified using the dominant primer pair, and for the DNA not derived from the sturgeon germplasm of interest, there was no product of amplification using the dominant primer pair. Specifically, the dominant primer pair may be the Hdau216p primer pair.

The test primer pair may be a co-dominant primer pair. Two primers of the co-dominant primer pair respectively correspond to forward and reverse sequences of the DNA molecule in the sturgeon genome. Therefore, for the DNA derived from the sturgeon germplasm of interest, its product of amplification obtained by using the co-dominant primer pair has the DNA molecule, while for the DNA not derived from the sturgeon germplasm, its product of amplification using the co-dominant primer pair does not have the DNA molecule. The size difference between the two products is 50 bp. Specifically, the co-dominant primer pair may be the Hdau195l primer pair.

The invention further provides an application of a substance used to test a specific molecular marker in identifying a sturgeon germplasm of interest; the sturgeon germplasm of interest is Huso dauricus or a hybrid of Huso dauricus and other sturgeons; the specific molecular marker is the DNA molecule or more than 10 consecutive nucleotides therein or more than 3 consecutive nucleotides at a terminal thereof.

The invention further provides a method for identifying whether a test sturgeon germplasm is a sturgeon germplasm of interest, including the following steps: testing whether the genomic DNA of the test sturgeon has a specific molecular marker, and if so, determining that the test sturgeon is the sturgeon germplasm of interest, wherein the sturgeon germplasm of interest is Huso dauricus or a hybrid of Huso dauricus and other sturgeons, and the specific molecular marker is the DNA molecule or more than 10 consecutive nucleotides therein or more than 3 consecutive nucleotides at a terminal thereof.

The invention further provides a method for identifying whether a test sturgeon germplasm is a sturgeon germplasm of interest, including the following steps: using the Hdau216p primer pair to perform PCR amplification with the genomic DNA of a test sturgeon as a template, and if an amplification product of 216 bp is obtained, determining that the test sturgeon is the sturgeon germplasm of interest, wherein the sturgeon germplasm of interest is Huso dauricus or a hybrid of Huso dauricus and other sturgeons.

The invention further provides a method for identifying whether a test sturgeon germplasm is Huso dauricus, including the following steps: using the Hdau195l primer pair to perform PCR amplification with the genomic DNA of a test sturgeon as a template, and if only one PCR amplification product of 195 bp is obtained, determining that the test sturgeon is Huso dauricus.

The invention further provides a method for identifying whether a test sturgeon germplasm is a hybrid of Huso dauricus and other sturgeons, including the following steps: using the Hdau195l primer pair to perform PCR amplification with the genomic DNA of a test sturgeon as a template, and if two PCR amplification products of 195 bp and 145 bp respectively are obtained, determining that the test sturgeon is a hybrid of Huso dauricus and other sturgeons.

The invention further provides the Hdau216p primer pair and/or the Hdau195l primer pair.

The invention further provides application of the Hdau216p primer pair and/or the Hdau195l primer pair in identifying a sturgeon germplasm of interest, wherein the sturgeon germplasm of interest is Huso dauricus or a hybrid of Huso dauricus and other sturgeons.

The Hdau216p primer pair consists of a primer of the sequence of SEQ ID NO: 4 and a primer of the sequence of SEQ ID NO: 5.

The Hdau1951p primer pair consists of a primer of the sequence of SEQ ID NO: 6 and a primer of the sequence of SEQ ID NO: 7.

Specifically, the amplification product of 216 bp is (a1) or (a2) as follows:
(a1) is the sequence of SEQ ID NO: 2; and
(a2) differs from the sequence of SEQ ID NO: 2 by less than 3 nucleotides.

Specifically, the amplification product of 195 bp is (b1) or (b2) as follows:
(b1) is the sequence of SEQ ID NO: 3; and
(b2) differs from the sequence of SEQ ID NO: 3 by less than 3 nucleotides.

Illustratively, the other sturgeons are Acipenser schrenckii, Siberian Acipenser, Acipenser gueldenstaedti, Acipenser ruthenus, Huso huso or Acipenser stellatus.

At present, there are no molecular markers that can be used to accurately identify purebreds and hybrids of Huso dauricus. The invention takes the five species of sturgeons (Acipenser schrenckii, Huso dauricus, Siberian Acipenser, Acipenser gueldenstaedti, and Acipenser ruthenus) that are mainly cultured in China as the research objects. By conducting comparative analysis of whole-genome resequencing data of the five sturgeons, a 50 bp InDel mutation region unique to Huso dauricus is screened out. Based on this specific insert fragment, a dominant/co-dominant germplasm-specific molecular marker is developed for the identification of Huso dauricus and its hybrids. The dominant molecular marker Hdau216p can be used to identify Huso dauricus and hybrids of Huso dauricus blood. According to the length specificity of PCR amplification products of Huso dauricus and other sturgeons, the co-dominant molecular marker Hdau195l can be used to quickly identify purebreds of Huso dauricus and corresponding hybrids by virtue of a PCR. The invention realizes the rapid identification of purebreds and hybrids of Huso dauricus by virtue of a PCR.

### BRIEF DESCRIPTION OF THE SEVERAL VIEWS OF THE DRAWINGS

FIG. 1 is a schematic diagram of the design principle of the Hdau216p primer pair.

FIG. 2 is an electrophoresis pattern after amplification using the Hdau216p primer pair.

FIG. 3 is a schematic diagram of the design principle of the Hdau195l primer pair.

FIG. 4 is an electrophoresis pattern after amplification using the Hdau195l primer pair.

### DETAILED DESCRIPTION OF THE INVENTION

The invention will be described in further detail below in conjunction with specific embodiments. The examples given are only for illustrating the invention and are not intended to limit the scope of the invention. The examples provided below can serve as a guide for those of ordinary skill in the art to make further improvements, and do not limit the invention in any way.

The experimental methods in the following examples, unless otherwise specified, are all conventional methods and are carried out in accordance with the techniques or conditions reported in the field or in accordance with product instructions. Unless otherwise specified, all the materials, reagents, etc., used in the following embodiments can be commercially available.

### Example 1 Discovery of InDel Marker

1. On the premise of ensuring the diversity of the population as much as possible, the fin tissues of 30 individuals from each of the five species of sturgeons (Acipenser schrenckii, Huso dauricus, Siberian Acipenser, Acipenser ruthenus, and Acipenser gueldenstaedti) were preserved in 95% ethanol and subjected to DNA extraction using a DNeasy Blood & Tissue Kit (NO. 69504) from Qiagen Company of Germany according to its instruction manual.

2. Equal amounts of DNAs obtained in step 1 were mixed in equal amount, an insert fragment of approximately 350 bp was prepared using an Illumina Hiseq 2000 platform from Novogene Company, and whole-genome resequencing was also performed.

3. Low-quality data and connectors in the original data were filtered using Fastp software. The raw data after quality control were aligned to the reference genome (ASM1064508v1) of Acipenser ruthenus using bwa with default parameters, and then a MarkDuplicates module in GATK software was used to remove potential PCR repetitive sequences. After alignment, a Haplotypecaller module in GATK software was used to identify genomic variant loci, and then all GVCF files were merged to identify and filter InDel variants to obtain high-quality InDel loci throughout the genome. A custom Python script was then used to screen the germplasm-specific homozygous InDel loci (sequencing depth greater than 10, InDel length greater than 20 and less than 60) in Huso dauricus in the results as candidate loci, and JBrowse was used to perform visual manual verification of alignment files.

4. Founded was a 50 bp small-fragment insertion mutation (i.e., the specific InDel marker in the invention) with germplasm specificity of Huso dauricus, located at position NC_048359.1:5385908 in the reference genome of Acipenser ruthenus, where the insertion of this fragment was detected in all 69 reads of Huso dauricus, but the data of other sturgeons did not have this insertion mutation here. Therefore, this locus was used as a candidate germplasm-specific variant locus of Huso dauricus.

The specific InDel marker is shown as the sequence of SEQ ID NO: 1.

### Example 2 Design of Test Primers on the Basis of InDel Marker

For the candidate InDel locus, 300 bp genome (Acipenser ruthenus genome) fragments in forward and reverse sequences of the variant locus were extracted. Primers were designed for candidate sequences and non-specific amplification was avoided. For the design of dominant primers, try to design the primer at one terminal completely or mostly in the insertion mutation sequence to ensure the amplification specificity of this pair of primers in the species of interest. For the design of co-dominant primers, try to design forward and reverse primers at two terminals of the insertion mutation to ensure the specificity of the length of the amplified fragment in the species of interest.

First, the primers were initially verified using part of sturgeon DNA samples used for sequencing. The primers that passed the verification were further verified by PCR using DNA samples from seven sturgeon species (Acipenser schrenckii, Huso dauricus, Siberian Acipenser, Acipenser ruthenus, Acipenser gueldenstaedti, Huso huso, and Acipenser stellatus) and hybrid sturgeons including the species of interest other than the sequencing samples.

The finally obtained dominant primers (named Hdau216p primer pair) are as follows:

forward primer:
(SEQ ID NO: 4)
CCCGGGGCACAAACATATC;
reverse primer:
(SEQ ID NO: 5)
CAAACAACAAGGAACATTGATACTG

The schematic diagram of the design principle of the Hdau216p primer pair is shown in FIG. 1. One end of the primer is designed in the insertion mutation of Huso dauricus, so the dominant primer has specific amplification in Huso dauricus but not in other sturgeons.

The finally obtained co-dominant primers (named Hdau195l primer pair) are as follows:

forward primer:
(SEQ ID NO: 6)
AAACATTTGTCACTTAGGGTTGCCA;
reverse primer:
(SEQ ID NO: 7)
TAACTCGTGCTCATTGTACRGGAA;

R represents A or G.

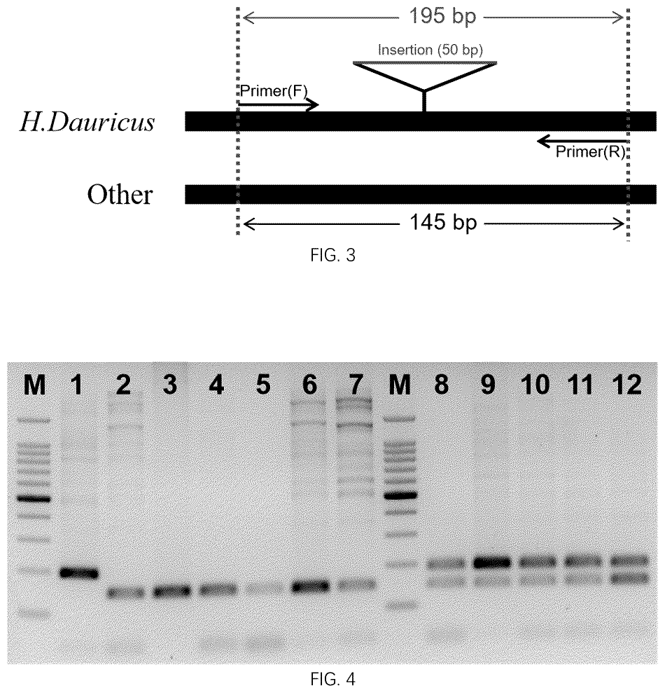

The schematic diagram of the design principle of the Hdau195l primer pair is shown in FIG. 3. Amplification was found in Huso dauricus and a specific amplification product of 195 bp was obtained, and in other sturgeons amplification was found and an amplification product of 145 bp was obtained.

### Example 3 Germplasm Identification Based on InDel Marker

Samples to be tested: Huso dauricus, Acipenser schrenckii, Siberian Acipenser, Acipenser gueldenstaedti, Acipenser ruthenus, Huso huso, Acipenser stellatus, hybrid offspring of Acipenser schrenckii and Huso dauricus [Acipenser schrenckii (♀)×Huso dauricus (♂)], hybrid offspring of Huso dauricus and Acipenser schrenckii [Huso dauricus (♀)×Acipenser schrenckii (♂)], hybrid offspring of Acipenser ruthenus and Huso dauricus [Acipenser ruthenus (♀)×Huso dauricus (♂)], hybrid offspring of Acipenser baeri and Huso dauricus [Acipenser baeri (♀)×Huso dauricus (♂)], and hybrid offspring of Acipenser gueldenstaedti and Huso dauricus [Acipenser gueldenstaedti (♀)×Huso dauricus (♂)].

1. The fin tissues of the samples to be tested were taken for genomic DNA extraction.

2. The Hdau216p primer pair or the Hdau195l primer pair was used for PCR amplification with the genomic DNA obtained in step 1 as a template.

Reaction system (20 μl) of PCR amplification, including: 0.5 μM forward primer, 0.5 μM reverse primer, 50 ng of template DNA, 10 μl of PrimeSTAR® Max DNA Polymerase (2×), and the balance of water. PrimeSTAR® Max DNA Polymerase (2×): Takara company.

The reaction procedure for PCR amplification was as follows:
Predenaturation: 94° C. 5 min;
35 cycles: 94° C. 30 s, 66° C. 30 s, 72° C. 10 s;
Final extension: 72° C. 10 min.

3. 3% agarose gel electrophoresis was performed on the PCR amplification product obtained in step 2, and the agarose gel was scanned by using ImageLab software.

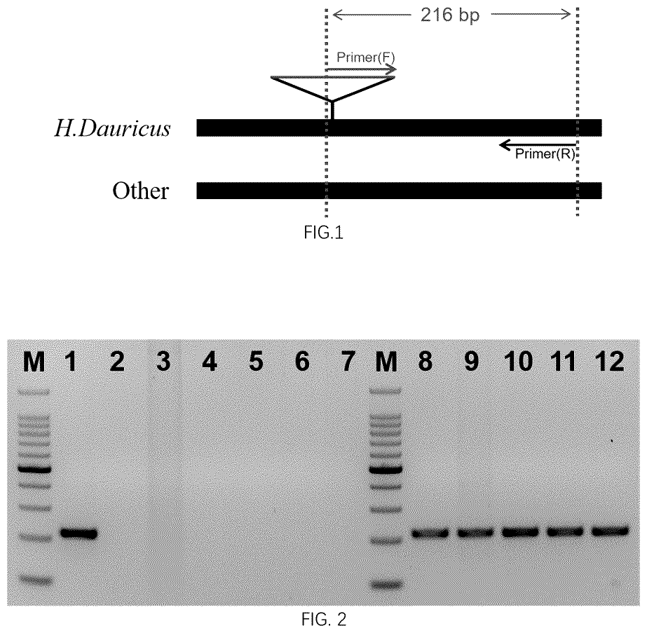

The electrophoresis pattern in the case of the Hdau216p primer pair is shown in FIG. 2. In FIG. 2, M: 100 bp DNA ladder; 1: Huso dauricus; 2: Acipenser schrenckii; 3: Siberian Acipenser; 4: Acipenser gueldenstaedti; 5: Acipenser ruthenus; 6: Huso huso; 7: Acipenser stellatus; 8: Acipenser schrenckii (♀)×Huso dauricus (♂); 9: Huso dauricus (♀)×Acipenser schrenckii (♂) 10: Acipenser ruthenus (♀)×Huso dauricus (♂); 11: Acipenser baeri (♀)×Huso dauricus (♂) 12: Acipenser gueldenstaedti (♀)×Huso dauricus (♂). Huso dauricus, the hybrid offspring of Acipenser schrenckii and Huso dauricus [Acipenser schrenckii (♀)×Huso dauricus (♂)], the hybrid offspring of Huso dauricus and Acipenser schrenckii [Huso dauricus (♀)×Acipenser schrenckii (♂)], the hybrid offspring of Acipenser ruthenus and Huso dauricus [Acipenser ruthenus (♀)×Huso dauricus (♂)], hybrid offspring of Acipenser baeri and Huso dauricus [Acipenser baeri (♀)×Huso dauricus (♂)], and the hybrid offspring of Acipenser gueldenstaedti and Huso dauricus [Acipenser gueldenstaedti (♀)×Huso dauricus (♂)] all showed specific amplification products. After sequencing verification, the specific amplification products of all samples were 216 bp. The sequencing result of one of the samples is shown as the sequence of SEQ ID NO: 2 (the sequencing results of other samples differ from the sequence of SEQ ID NO: 2 by less than 3 nucleotides). No amplification products were obtained for Acipenser schrenckii, Siberian Acipenser, Acipenser gueldenstaedti, Acipenser ruthenus, Huso huso, and Acipenser stellatus.

The results in the case of the Hdau195l primer pair are shown in FIG. 4. In FIG. 4, M: 100 bp DNA ladder; 1: Huso dauricus; 2: Acipenser schrenckii; 3: Siberian Acipenser; 4: Acipenser gueldenstaedti; 5: Acipenser ruthenus; 6: Huso huso; 7: Acipenser stellatus; 8: Acipenser schrenckii (♀)×Huso dauricus (♂); 9: Huso dauricus (♀)×Acipenser schrenckii (♂); 10: Acipenser ruthenus (♀)×Huso dauricus (♂); 11: Acipenser baeri (♀)×Huso dauricus (♂); 12: Acipenser gueldenstaedti (♀)×Huso dauricus (♂). The amplification product of Huso dauricus showed a single band, which was verified by sequencing to be 195 bp and contained the specific InDel marker having the sequence of SEQ ID NO: 1. The amplification products of the hybrid offspring of Acipenser schrenckii and Huso dauricus [Acipenser schrenckii (♀)×Huso dauricus (♂)], the hybrid offspring of Huso dauricus and Acipenser schrenckii [Huso dauricus (♀)×Acipenser schrenckii (♂)], the hybrid offspring of Acipenser ruthenus and Huso dauricus [Acipenser ruthenus (♀)×Huso dauricus (♂)], hybrid offspring of Acipenser baeri and Huso dauricus [Acipenser baeri (♀)×Huso dauricus (♂)], and the hybrid offspring of Acipenser gueldenstaedti and Huso dauricus [Acipenser gueldenstaedti (♀)×Huso dauricus (♂)] all showed two bands. After sequencing verification, one amplification product was 195 bp (and all had the specific InDel marker of the sequence of SEQ ID NO: 1), and the other amplification product was 145 bp (and all lacked the specific InDel marker shown in the sequence of SEQ ID NO: 1). The sequencing result of the 195 bp amplification product of one of the samples is the sequence of SEQ ID NO: 3 (the sequencing results of 195 bp amplification products of other samples differ from the sequence of SEQ ID NO: 3 by less than 3 nucleotides). The amplification products of Acipenser schrenckii, Siberian Acipenser, Acipenser gueldenstaedti, Acipenser ruthenus, Huso huso, and Acipenser stellatus showed a single band, which was verified by sequencing to be 145 bp and lacked the specific InDel marker of the sequence of SEQ ID NO: 1.

The invention has been described in detail above. For those skilled in the art, the invention can be implemented in a wider range under equivalent parameters, concentrations and conditions without departing from the spirit and scope of the invention and without performing unnecessary experiments. Although specific embodiments of the invention have been shown, it should be understood that further modifications can be made to the invention. In summary, based on the principles of the invention, the present application is intended to include any changes, uses, or improvements to the invention, including changes that depart from the scope disclosed in the present application and are made using conventional techniques known in the art. Some essential features may be applied within the scope of the appended Claims below.

We claim:

1. A DNA molecule of SEQ ID NO: 1.

2. A method for identifying a sturgeon germplasm of interest according to claim 1, comprising
applying the DNA molecule of claim 1 or more than 10 consecutive nucleotides therein or more than 3 consecutive nucleotides at a terminal thereof as a sturgeon germplasm molecular marker,
identifying a sturgeon germplasm of interest by using the sturgeon germplasm molecular marker, wherein the sturgeon germplasm of interest is Huso dauricus or a hybrid of Huso dauricus and other sturgeons.

3. A test primer pair developed on the basis of the DNA molecule according to claim 1 or more than 10 consecutive nucleotides therein or more than 3 consecutive nucleotides at a terminal thereof.

4. A method of using a substance to test a specific molecular marker in identifying a sturgeon germplasm of interest according to claim 1,
wherein the sturgeon germplasm of interest is Huso dauricus or a hybrid of Huso dauricus and other sturgeons, and
the specific molecular marker is the DNA molecule according to claim 1 or more than 10 consecutive nucleotides therein or more than 3 consecutive nucleotides at a terminal thereof.

5. A method for identifying a sturgeon germplasm of interest according to claim 1, comprising:
testing genomic DNA of a test sturgeon for a specific molecular marker, and
determining the test sturgeon is the sturgeon germplasm of interest,
wherein the sturgeon germplasm of interest is Huso dauricus or a hybrid of Huso dauricus and other sturgeons, and
the specific molecular marker is the DNA molecule according to claim 1 or more than 10 consecutive nucleotides therein or more than 3 consecutive nucleotides at a terminal thereof.

6. The method of claim 5, further comprising
performing PCR amplification with the genomic DNA of the test sturgeon as a template by using a Hdau216p primer pair,
determining that the test sturgeon is the sturgeon germplasm of interest by obtaining an 216 bp amplification product by the Hdau216p primer pair,
wherein the sturgeon germplasm of interest is Huso dauricus or a hybrid of Huso dauricus and other sturgeons, and the Hdau216p primer pair consists of a primer of SEQ ID NO: 4 and a primer of SEQ ID NO: 5.

7. The method of claim 5, further comprising
performing PCR amplification with the genomic DNA of the test sturgeon as a template by using a Hdau195l primer pair,
determining that the test sturgeon is the sturgeon germplasm of interest by obtaining an 195 bp amplification product by the Hdau195l primer pair,
wherein the sturgeon germplasm of interest is Huso dauricus, and the Hdau195l primer pair consists of a primer of SEQ ID NO: 6 and a primer of SEQ ID NO: 7.

8. The method of claim 5, further comprising
performing PCR amplification with the genomic DNA of the test sturgeon as a template by using a Hdau195l primer pair,
determining that the test sturgeon is the sturgeon germplasm of interest by obtaining a 195 bp amplification product and a 145 bp amplification product, respectively, by the Hdau195l primer pair,
wherein the sturgeon germplasm of interest is a hybrid of Huso dauricus and other sturgeons, and the Hdau195l primer pair consists of a primer of SEQ ID NO: 6 and a primer of SEQ ID NO: 7.

9. A set of primer pairs for identifying a sturgeon germplasm of interest, comprising
a Hdau216p primer pair consisting of a primer of SEQ ID NO: 4 and a primer of SEQ ID NO: 5, and
a Hdau195l primer pair consisting of a primer of SEQ ID NO: 6 and a primer of SEQ ID NO: 7.

10. A method for identifying a sturgeon germplasm of interest according to claim 9, comprising
using the set of primer pairs of claim 9,
wherein the sturgeon germplasm of interest is Huso dauricus or a hybrid of Huso dauricus and other sturgeons.


SEQUENCE LISTING (7 sequences)

SEQ ID NO: 1 - DNA, 50 bp, synthetic construct
  source 1..50: mol_type = other DNA
ttttctctggatcgtcctggttttgaggaaggtgtcccggggcacaaaca

SEQ ID NO: 2 - DNA, 216 bp, synthetic construct
  source 1..216: mol_type = other DNA
cccggggcacaaacatatcctgctttttaatcttacccgtttataatcttacgcttccacgtccaagtagataagatcactacagcaacactgcaaagacattcctgtacaatgagcacgagttaatcatcaagaaaatattactgcaatagacgcacaatatgaatgggttgtcagtactttaatagtaacagtatcaatgttccttgttgtttg

SEQ ID NO: 3 - DNA, 195 bp, synthetic construct
  source 1..195: mol_type = other DNA
aaacatttgtcacttagggttgccaagtgtccctgttttctctggatcgtcctggttttgaggaaggtgtcccggggcacaaacatatcctgctttttaatcttacccgtttataatcttacgcttccacgtccaagtagataagatcactacagcaacactgcaaagacattcctgtacaatgagcacgagtta

SEQ ID NO: 4 - DNA, 19 bp, synthetic construct
  source 1..19: mol_type = other DNA
cccggggcacaaacatatc

SEQ ID NO: 5 - DNA, 25 bp, synthetic construct
  source 1..25: mol_type = other DNA
caaacaacaaggaacattgatactg

SEQ ID NO: 6 - DNA, 25 bp, synthetic construct
  source 1..25: mol_type = other DNA
aaacatttgtcacttagggttgcca

SEQ ID NO: 7 - DNA, 24 bp, synthetic construct
  source 1..24: mol_type = other DNA
taactcgtgctcattgtacrggaa